(19) United States
(12) Patent Application Publication
(10) Pub. No.: US 2006/0201504 A1
(43) Pub. Date: Sep. 14, 2006

(54) High-flow oxygen delivery system and methods of use thereof

(76) Inventors: Aneesh B. Singhal, Boston, MA (US); A. Gregory Sorensen, Lexington, MA (US); Dean Hess, Danvers, MA (US); Eng H. Lo, Auburndale, MA (US); Walter J. Koroshetz, Milton, MA (US)

Correspondence Address: CLARK & ELBING LLP, 101 FEDERAL STREET, BOSTON, MA, 02110, US

(21) Appl. No.: 11/075,990

(22) Filed: Mar. 8, 2005

(51) Int. Cl. A61M 16/00; A62B 9/00

(52) U.S. Cl. 128/204.18; 128/205.22

(57) ABSTRACT

A device and method of its use for the treatment or prevention of an acute ischemic condition by administering high concentrations of oxygen or an oxygen- containing gas at normobaric pressure and a flow rate of 10 L/min or greater.

### BACKGROUND OF THE INVENTION

Organ ischemia occurs when blood flow to an organ is interrupted, usually by a blood clot or a severe drop in blood pressure. Organ ischemia can also occur when tissue compartmental pressure rises to a level that compromises blood flow (for example, brain ischemia in patients with stroke or head injury and raised intracranial pressure).

When blood flow is interrupted, cells are deprived of oxygen and nutrients and begin to die within minutes. In humans, there is usually a core of dead tissue (“infarct”), surrounded by tissue that is still alive but at high risk of death (“penumbra”) due to decreased blood flow, oxygen, and perturbation of biochemical, metabolic, and cellular pathways. A major goal of acute ischemia treatment is the re-establishment of blood flow (“reperfusion”), thereby re-establishing oxygen and nutrient supply to tissue. In the case of ischemic brain infarction (stroke), or ischemia of the brain or spinal cord, re-establishment of blood flow is accomplished by first lysing the clot using a clot-busting drug, such as tissue plasminogen activator (tPA). In the case of stroke or cardiac ischemia, this may also entail angioplasty and stenting. Another approach to treat organ ischemia involves the administration of drugs to protect the ischemic organ. For example, in the case of acute stroke, pharmacologic agents (i.e., “neuroprotective” drugs) that impede the cascade of biochemical, metabolic, and cellular events that lead to cell death after ischemia can be administered. The time frame for reperfusion and/or delivery of organ-protective agents is, for most instances of organ ischemia, narrow.

In patients with cerebral ischemia due to raised intracranial pressure, cerebral blood flow can be augmented by decreasing the amount of brain edema, using, e.g., drugs such as hypertonic saline or mannitol, or by surgical maneuvers such as hemicraniectomy. The medical treatments have transient effects, cannot be administered indefinitely, and are limited by a “rebound” increase in intracranial pressure when therapy is withdrawn. Surgical treatments, i.e. hemicraniectomy, are considered radical and have not been proven to improve outcome. Hence at this time there is no satisfactory means to prevent brain ischemia due to raised intracranial pressure.

Hyperbaric oxygen therapy is the treatment of the entire body with 100% oxygen at a pressure higher than sea-level pressure (i.e., greater than 1 atmosphere absolute (ATA) and usually 2-3 times ATA) while inside a treatment chamber. Hyperbaric oxygen therapy has long been accepted as the definitive treatment for decompression illness (the “bends”), a complication of diving, and much of the terminology and structure of this therapy reflects that history. Hyperbaric oxygen therapy may be carried out in either a monoplace or multiplace chamber. The former accommodates a single patient and the entire chamber is pressurized with 100% oxygen, which the patient breathes directly. The latter holds two or more people (patients, support personnel, observers) and the chamber is pressurized with air; the patients breathe 100% oxygen by masks, head hoods, or endotracheal tube. The purpose of the therapy is to provide increased amounts of oxygen to the body.

Hyperbaric oxygen therapy has been used to treat several disorders including, e.g., decubitus ulcers, radiation necrosis, acute carbon monoxide poisoning, acute gas embolism, gas gangrene, refractory osteomyelitis, crush injuries with acute traumatic ischemia, acute cyanide poisoning, acute cerebral edema, thermal bums, bone grafting, acute carbon tetrachloride poisoning, fracture healing, multiple sclerosis, sickle cell anemia, and numerous other conditions. While there has been some success in treating patients suffering acute ischemic events (including stroke, myocardial infarction, and brain trauma) using hyperbaric oxygen therapy, this therapy has several limitations including cost and inconvenience to the patient, who must remain in a hyperbaric oxygen chamber typically for a period of about an hour, which limits access of the patient to medical care and testing.

A potential therapy for the treatment of acute organ ischemia is the administration of low volume (i.e., <5 L/min) inhaled oxygen at room pressure. To date, in the case of acute stroke, the effect of delivering oxygen at room pressure has been evaluated in only a single observational study where oxygen was administered at a rate of 3 L/min for a period of twenty-four hours after stroke. This study showed no benefit with supplemental oxygen. Thus, there remains a need for a method and device that can administer oxygen at room pressure, in high enough concentrations to successfully treat or prevent organ ischemia. Delivering high concentrations of oxygen with such a device may also be a means to safely extend the narrow time window for treating patients experiencing or suspected of having an acute ischemic condition with clot-busting drugs such as tPA, with mechanical devices such as angioplasty or stenting, or with “protective” agents such as neuroprotective or cardioprotective drugs.

### SUMMARY OF THE INVENTION

In one aspect, the invention features an apparatus for supplying a high concentration of oxygen to the respiratory tract of a patient with a known or suspected acute condition associated with ischemia (e.g., stroke, subarachnoid hemorrhage, brain hemorrhage, hemorrhagic stroke, brain trauma, head injury, head trauma, a seizure, a headache disorder (e.g., migraine or cluster headache), cardiovascular disease (e.g., myocardial infarction, heart disease, coronary artery disease, congestive heart failure, cardiac valvular disease, cardiac arrhythmia, and cardiac arrest), tissue organ engraftment rejection, sequelae of ischemic reperfusion injury, retinal ischemia (e.g., diabetic retinopathy, central retinal artery or vein occlusion, stenosis of the carotid artery, and sickle cell retinopathy), retinal detachment, retinal tearing, gastrointestinal ischemia (e.g., ischemic bowel, ischemic colitis, and mesenteric ischemia), kidney ischemia, peripheral ischemia (e.g., acute peripheral vascular ischemia, atherosclerosis, peripheral arterial occlusive disease, thromboembolic disease, and thromboangiitis obliterans (Buerger's disease)), or other organ ischemia, that includes a source of oxygen and means for delivering the oxygen (e.g., a facemask coupled to the source of oxygen) at a flow rate of 10 L/min or greater at room pressure or normal or near-normal atmospheric pressure (e.g., between about 0.5 and 1.5 ATA, preferably about 1 ATA), to the respiratory system of a human patient. The apparatus is adaptable to be portable, MRI-compatible, or both.

In preferred embodiments of the above aspect, the oxygen delivered to the respiratory system of the patient is between about 90% and 100% oxygen, preferably about 95% oxygen, and more preferably about 100% oxygen. The oxygen delivered to the patient is provided from a container that contains about 90% or greater oxygen, more preferably about 95% oxygen, and most preferably about 100% oxygen. In other preferred embodiments, the flow rate of the oxygen is at least 25 L/min or higher, preferably between 25 L/min and 60 L/min, more preferably between about 30 to 40 L/min, and most preferably about 35 L/min or about 40 L/min, and the oxygen is delivered at normal or near-normal atmospheric pressure. In another embodiment, the flow rate is higher than the patient's maximal inspiratory flow rate.

In another preferred embodiment of the above aspect, the apparatus achieves a flow rate that raises the partial pressure of a patient's arterial oxygen (paO₂) above about 200 mm Hg when the oxygen is delivered at normal or near-normal atmospheric pressure.

In preferred embodiments of the above aspect, the materials used to manufacture the high-flow oxygen delivery apparatus are magnetic resonance (MR)-compatible. For example, the materials can be non-metallic (plastic or silicon), non-ferromagnetic, and non-paramagnetic. In preferred embodiments, the materials are aluminum, brass, or stainless steel. MR-compatibility is a significant advantage for the treatment of certain ischemia conditions, in particular stroke, for which an MR image is typically obtained shortly after the patient is admitted to the hospital; administration of normobaric high-flow oxygen therapy (“NBO”) to the patient can continue uninterrupted while MR imaging is being carried out. For the same reason, i.e., assuring uninterrupted administration of cell-saving oxygen during MR imaging, the apparatus of the invention is preferably portable, allowing it to accompany the patient in the MR machine. Portability also allows the oxygen delivery device to accompany patients throughout the hospital and also permits pre- and post-hospitalization home use, and use in an ambulance to and from the hospital.

The apparatus can also, optionally, include humidifying means for humidifying the oxygen from the source, end-tidal capnometer sensor means or similar means for measuring exhaled carbon dioxide levels and thereby sensing hypoventilation, an automatic shut-off device so as to safeguard against hypoventilation, and gas demand valve means that allows the flow of oxygen solely during inspiration of the patient. If oxygen is delivered via a facemask, it is preferred that the facemask be tight-fitting, e.g., it can be secured using elastic straps, VELCRO®, or other means to ensure no loss of oxygen during delivery and to reduce the potential for room air dilution.

A second aspect of the invention features a method of reducing acute tissue damage in a human patient that has or is likely to have acute organ infarction, other than a stroke patient, by delivering oxygen to the respiratory system of the patient at a flow rate of 25 L/min or greater at normal or near-normal atmospheric pressure. In a preferred embodiment, the oxygen delivered to the respiratory system of the patient is between about 90% and 100% oxygen, preferably about 95% oxygen, and more preferably about 100% oxygen. The oxygen delivered to the patient is provided from a container that contains about 90% or greater oxygen, more preferably about 95% oxygen, and most preferably about 100% oxygen. In other preferred embodiments, the flow rate of the oxygen is at least 25 L/min or higher, preferably between about 25 L/min and about 60 L/min, more preferably about 30 to 40 L/min, and most preferably about 35 L/min or about 40 L/min, and the oxygen is delivered at normal or near-normal atmospheric pressure (e.g., between about 0.5 and 1.5 ATA, preferably about 1 ATA). In another embodiment, the flow rate is higher than the patient's maximal inspiratory flow rate, or is a flow rate that raises the paO₂ value above about 200 mm Hg. In yet another preferred embodiment, the method includes delivering oxygen using the apparatus of the first aspect. The apparatus is, preferably, adaptable to be portable, MRI-compatible, or both, but neither of these adaptations is required for treatment.

In preferred embodiments of the second aspect, the acute condition to be treated is subarachnoid hemorrhage, brain hemorrhage, brain trauma, head injury, head trauma, a seizure, a headache disorder (e.g., migraine or cluster headache), cardiovascular disease (e.g., myocardial infarction, heart disease, coronary artery disease, congestive heart failure, cardiac valvular disease, cardiac arrhythmia, or cardiac arrest), tissue organ engraftment rejection, sequelae of ischemic reperfusion injury, retinal ischemia (e.g., diabetic retinopathy, central retinal artery or vein occlusion, stenosis of the carotid artery, or sickle cell retinopathy), retinal detachment, retinal tearing, gastrointestinal ischemia (e.g., ischemic bowel, ischemic colitis, or mesenteric ischemia), kidney ischemia, peripheral ischemia (e.g., acute peripheral vascular ischemia, atherosclerosis, peripheral arterial occlusive disease, thromboembolic disease, or thromboangiitis obliterans (Buerger's disease)), or other organ ischemia. Preferably, the method further involves the delivery of 100% oxygen to the patient. In another preferred embodiment, the oxygen is delivered to the patient at a flow rate that raises the paO₂ value above about 200 mm Hg. In another preferred embodiment, the oxygen is delivered to the patient using delivery means (e.g., a facemask coupled to the source of oxygen).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a plan view of a high-flow oxygen delivery apparatus of the invention.

FIG. 2 is a plan view of a facemask for use with the device of FIG. 1.

FIG. 3 is a plan view of a portion of the device of FIG. 1 that includes a humidifier and a vessel containing a humidifying liquid, e.g., water.

FIGS. 4A-4C are graphs showing (A) NIHSS scores, (B) Percent change in relative stroke lesion volumes, and (C) Penumbral salvage, or the ratio of acutely hypoperfused tissue salvaged from infarction [(MTT at time-point 1)-(infarct volume at later time point)] to the acute tissue at risk for infarction [(MTT at time-point 1)-(DWI at time-point 1)]. Controls, white bars; NBO, black bars; mean±SD. *P<0.01 versus controls.

FIGS. 5A-5C are serial MRI findings in a patient with cardio-embolic right-MCA stroke treated with NBO for 8 hours. FIG. 5A is the baseline (pre-NBO) MRI, 13.1 hours post-symptom onset, showing a large DWI lesion, a larger MTT lesion, and MCA-occlusion (arrow) on head MRA. FIG. 5B is a second MRI after 3.75 hours (during-NBO) showing 36% reduction in the DWI lesion, stable MTT deficit, and persistent MCA-occlusion. FIG. 5C is a third MRI after 24 hours (post-NBO) showing reappearance of DWI abnormality in some areas of previous reversal; MTT image shows partial reperfusion (39% MTT volume reduction, mainly in the ACA territory); MRA shows partial MCA recanalization.

FIG. 6A presents a 24 hour DWI image with color-coded overlays showing fate of individual ADC voxels from 0-24 hours, in three NBO (a-c) and two control (d, e) patients. Patient b is the same as in FIGS. 5A-5C. Voxels undergoing temporary early ADC-reversal (green) and sustained early ADC-reversal (blue) are present mainly in the lesion periphery, and clearly evident in all NBO patients. The few voxels undergoing late ADC-reversal (cyan) do not have a distinct distribution pattern. Voxels showing no change (red) predominate in the center of the DWI lesions in both groups, and voxels showing progressive ischemia (yellow) are most evident in the Control patients.

FIG. 6B is a bar-graph showing the fate of individual voxels (mean±SD) on ADC maps from 0-24 hours. Controls, white bars; NBO, black bars.

FIGS. 7A-7C are bar-graphs showing rCBV (A), rCBF (B), and rMTT (C) (normalized values, mean±SD) from brain regions showing visible MTT prolongation at baseline. These parameters were not significantly different between groups at any time-point, however in the NBO group, rCBV and rCBF increased significantly (*p<0.01, **p<0.05) over baseline values. Controls, white bars; NBO, black bars.

### DETAILED DESCRIPTION OF THE INVENTION

Normobaric high-flow oxygen therapy (NBO) can be used to salvage ischemic tissue resulting from an acute condition, such as that caused by ischemic stroke, subarachnoid hemorrhage, brain hemorrhage, hemorrhagic stroke, brain trauma, head injury, head trauma, a seizure, a headache disorder (e.g., migraine or cluster headache), cardiovascular disease (e.g., myocardial infarction, heart disease, coronary artery disease, congestive heart failure, cardiac valvular disease, cardiac arrhythmia, or cardiac arrest), tissue organ engraftment rejection, sequelae of ischemic reperfusion injury, retinal ischemia (e.g., diabetic retinopathy, central retinal artery or vein occlusion, stenosis of the carotid artery, or sickle cell retinopathy), retinal detachment, retinal tearing, gastrointestinal ischemia (e.g., ischemic bowel, ischemic colitis, or mesenteric ischemia), kidney ischemia, peripheral ischemia (e.g., acute peripheral vascular ischemia, atherosclerosis, peripheral arterial occlusive disease, thromboembolic disease, or thromboangiitis obliterans (Buerger's disease)), or other organ ischemia. The device and method safely treat or extend the time window for treating patients experiencing or suspected of having an acute ischemic condition. The method involves inhalation of oxygen at high concentrations (>80% concentration, preferably between about 90% to 100%, more preferably about 95%, and most preferably about 100%) and at high flow rates. For example, oxygen can be delivered using the device at a flow rate of about 10 L/min or greater (e.g., at least about 25 L/min or higher, preferably between about 25 L/min and 60 L/min, more preferably between about 30 to 40 L/min, and most preferably about 35 L/min or about 40 L/min). A flow rate of 40 L/min usually exceeds the maximum resting inspiratory flow rate of humans, so the patient essentially breathes 100% oxygen and/or easily achieves paO2 values greater than 200 mm Hg, which is known to be effective in reducing ischemic tissue damage.

By “normobaric” is meant at or about 1 ATA pressure (e.g., normobaric pressure includes pressures in the range of between 0.5 and 1.5 ATA). According to accepted definition, neither breathing 100% oxygen at 1 ATA pressure (normobaric oxygen) nor exposing discrete parts of the body to 100% oxygen (topical oxygen) constitute hyperbaric oxygen therapy.

Preferably, the patients are treated as soon as possible after onset of the acute condition associated with ischemia (e.g., stroke, subarachnoid hemorrhage, brain hemorrhage, hemorrhagic stroke, brain trauma, head injury, head trauma, a seizure, a headache disorder (e.g., migraine or cluster headache), cardiovascular disease (e.g., myocardial infarction, heart disease, coronary artery disease, congestive heart failure, cardiac valvular disease, cardiac arrhythmia, and cardiac arrest), tissue organ engraftment rejection, sequelae of ischemic reperfusion injury, retinal ischemia (e.g., diabetic retinopathy, central retinal artery or vein occlusion, stenosis of the carotid artery, and sickle cell retinopathy), retinal detachment, retinal tearing, gastrointestinal ischemia (e.g., ischemic bowel, ischemic colitis, and mesenteric ischemia), kidney ischemia, peripheral ischemia (e.g., acute peripheral vascular ischemia, atherosclerosis, peripheral arterial occlusive disease, thromboembolic disease, and thromboangiitis obliterans (Buerger's disease)), or other organ ischemia). Delivery of oxygen can be initiated at home, in the field (e.g., by paramedics), in the emergency,room, or in the hospital or nursing home using the high-flow oxygen delivery apparatus disclosed. Oxygen therapy can be administered until the time that the ischemic organ is successfully reperfused, or until any thrombolytic or protective agents (e.g., tPA or a neuroprotective drug) are administered. Treatment can be continued even following reperfusion. Treatment can be continued for about eight hours or longer, if necessary. The duration of oxygen therapy can be determined by the treating physician or nurse. The oxygen therapy can be initiated as the sole therapy for the acute ischemic condition, or as a supplemental therapy in conjunction with reperfusion treatments (e.g., thrombolytics, anticoagulants, or angioplasty/stenting), or the administration of a cytoprotective agent (e.g., a neuroprotective agent).

Structure

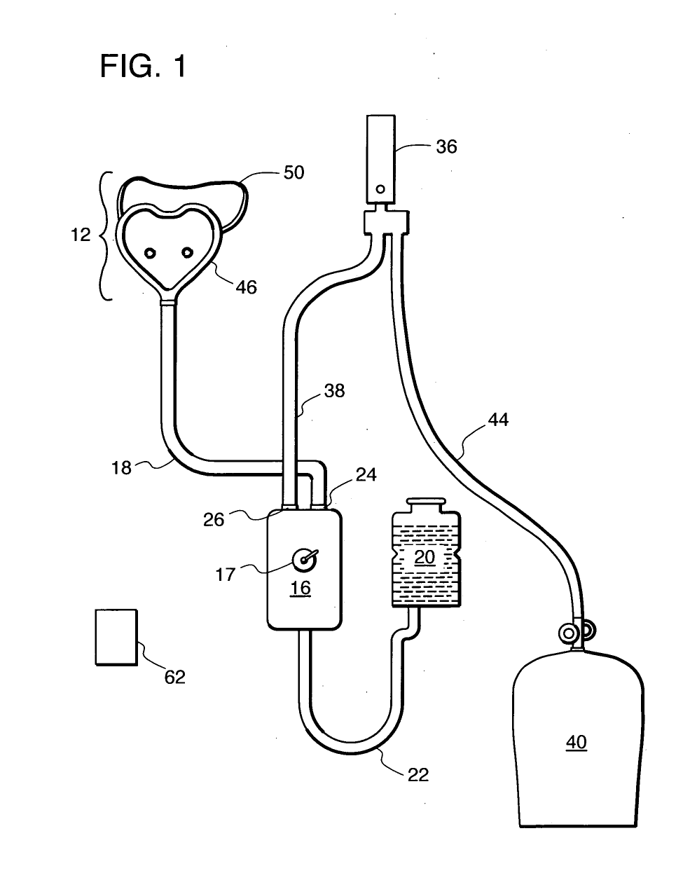

Referring to FIG. 1, high-flow oxygen delivery apparatus (10) includes facemask (12) coupled via medical grade wide-bore plastic tubing (18) to humidifier (16) at outlet supply port (24). Humidifier (16) is connected to vessel (20), which forms an internal volume for holding humidifying liquid, e.g., water, by medical grade plastic tubibg (22). Humidifier (16) also includes inlet supply port (26), which is coupled to flowmeter (36) by medical grade plastic tubing (38). Humidifier (16), vessel (20) and connecting tubibg (22) are optional, and facemask (12) can be connected directly to flowmeter (36). Flowmeter (36) is connected to cylinder (40) containing oxygen or an oxygen-containing gas via conventional medical grade tubing (44) or other conduit means for providing fluid (gas) communication between facemask (12), optional humidifier (16), and cylinder (40). Flowmeter (36) controls the rate at which gas flows from cylinder (40). The flow rate of gas is usually measured in liters per minute (LPM). High-flow oxygen delivery apparatus (10) also, optionally, includes oxygen arterial saturation sensor (62) for measuring oxygen saturation levels in the peripheral arteries of the patient. Cylinder (40) contains about 90% or greater oxygen, more preferably about 95% oxygen, and most preferably about 100% oxygen.

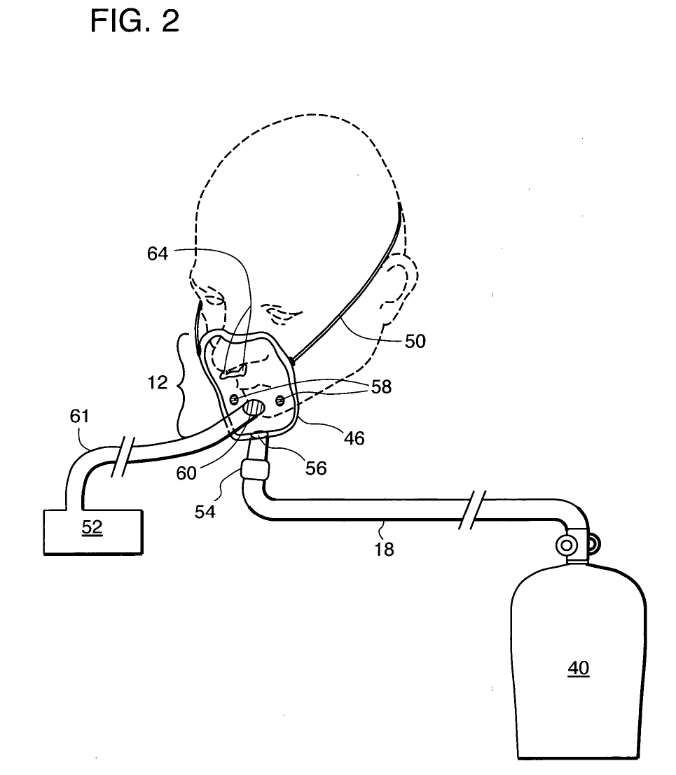

FIG. 2 shows facemask (12), which is in the form of cup-like body (46) and equipped with gas inhalation valve (56) and one-way gas exhalation valves (58). Facemask (12) includes adjustable elastic or VELCRO® strap (50), which is connected to the sides of body (46) to allow attachment of facemask (12) to the head of a patient. Optionally, facemask (12) includes end-tidal capnometer device (52), which is connected by medical grade tubing (61) to facemask (12) and which includes, at the facemask, capnometer sensor (60) at the mouth or capnometer sensor (64) at the nostrils. Capnometer device (52) analyzes carbon dioxide gases expired by the patient during air exchange in the lungs of the patient using high-flow oxygen delivery system (10), and may serve to shut off oxygen delivery if the expired carbon dioxide concentration becomes too high.

Also optionally connected to facemask (12) via medical grade tubing (18) is oxygen demand valve (54), which allows the flow of oxygen or oxygen-containing gas only during the inspiratory phase.

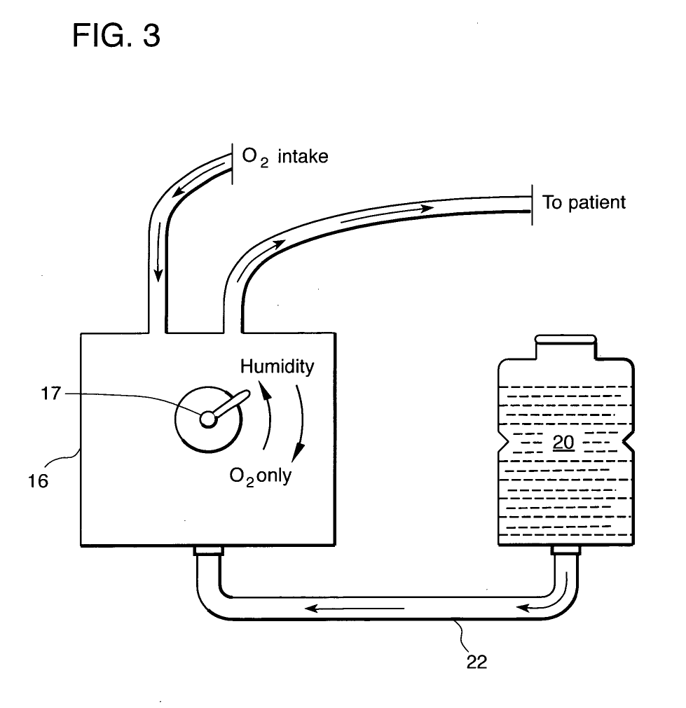

FIG. 3 shows humidifier (16) connected to vessel (20) containing a humidifying liquid, e.g., water. Humidifier (16) is designed to allow the user to switch the flow of oxygen by manipulating knob (17) to direct pure oxygen or oxygen-containing gas to the patient, or alternately, to rout oxygen or oxygen-containing gas (such as oxygen mixed with nitrogen) through humidifier (16) to provide humidified oxygen to the patient.

The medical grade plastic tubing (18, 22, 38, 44, 61) that connects the various components of the high-flow oxygen delivery apparatus (10) is removably secured to its respective component in a gas-tight fashion and allows a flow rate of oxygen of at least between about 5 liter/min and 40 liters/min, and preferably allowing a flow rate of up to 60 L/min.

Preferably, the materials used to manufacture the high-flow oxygen delivery apparatus (10) are magnetic resonance (MR)-compatible. For example, the materials are non-metallic (plastic or silicon), non-ferromagnetic, and non-paramagnetic. In preferred embodiments, the materials are aluminum, brass, or stainless steel. Manufacture of the device using MR-compatible materials facilitates the continued administration of oxygen to a patient using the device during magnetic resonance imaging (MRI).

Operation

High-flow oxygen delivery apparatus (10) provides effective concentrations of oxygen or oxygen-containing gas to the respiratory tract of a patient with a known or suspected acute condition associated with ischemia, e.g., stroke, subarachnoid hemorrhage, brain hemorrhage, hemorrhagic stroke, brain trauma, head injury, head trauma, a seizure, a headache disorder (e.g., migraine or cluster headache), cardiovascular disease (e.g., myocardial infarction, heart disease, coronary artery disease, congestive heart failure, cardiac valvular disease, cardiac arrhythmia, and cardiac arrest), tissue organ engraftment rejection, sequelae of ischemic reperfusion injury, retinal ischemia (e.g., diabetic retinopathy, central retinal artery or vein occlusion, stenosis of the carotid artery, and sickle cell retinopathy), retinal detachment, retinal tearing, gastrointestinal ischemia (e.g., ischemic bowel, ischemic colitis, and mesenteric ischemia), kidney ischemia, peripheral ischemia (e.g., acute peripheral vascular ischemia, atherosclerosis, peripheral arterial occlusive disease, thromboembolic disease, and thromboangiitis obliterans (Buerger's disease)), or other organ ischemia. High-flow oxygen delivery apparatus (10) can be modified for use during patient transportation (i.e., the device can be made portable) and for use during MRI (using, e.g,. MR-compatible materials, such as aluminum, brass, and stainless steel).

High-flow oxygen delivery apparatus (10) can be used to treat an ischemic condition in a patient directly, or as an adjunct therapy for use with other therapies. For example, if the patient suffers an ischemic stroke or myocardial infarction, high-flow oxygen delivery apparatus (10) can be used alone or in combination with, e.g., thrombolytic, neuroprotective, or cardioprotective drugs, or angioplasty/stenting, respectively.

High-flow oxygen delivery apparatus (10) delivers oxygen or an oxygen-containing gas at a high flow rate of between about 10 L/min and 60 L/min, preferably about 40 L/min, at normal atmospheric pressure (normobaric pressure) to treat a patient with a known or suspected condition associated with ischemia, e.g., stroke, subarachnoid hemorrhage, hemorrhagic stroke, brain hemorrhage, brain trauma, head injury, head trauma, a seizure, a headache disorder (e.g., migraine or cluster headache), cardiovascular disease (e.g., myocardial infarction, heart disease, coronary artery disease, congestive heart failure, cardiac valvular disease, cardiac arrhythmia, and cardiac arrest), tissue organ engraftment rejection, sequelae of ischemic reperfusion injury, retinal ischemia (e.g., diabetic retinopathy, central retinal artery or vein occlusion, stenosis of the carotid artery, and sickle cell retinopathy), retinal detachment, retinal tearing, gastrointestinal ischemia (e.g., ischemic bowel, ischemic colitis, and mesenteric ischemia), kidney ischemia, peripheral ischemia (e.g., acute peripheral vascular ischemia, atherosclerosis, peripheral arterial occlusive disease, thromboembolic disease, and thromboangiitis obliterans (Buerger's disease)), or other organ ischemia. For therapeutic purposes, flow rates of approximately 40 L/min or higher show efficacy for the treatment of ischemic conditions in humans. The flowmeter (36) is capable of delivering >10 L/min oxygen to a patient. Preferably, high-flow oxygen delivery apparatus (10) delivers about 85% or greater oxygen to the patient, preferably about 90% or greater oxygen, more preferably about 95% or greater oxygen, and most preferably about 100% oxygen.

Oxygen therapy should be initiated early, i.e., as soon as possible after onset of symptoms, and should be continued for at least 15 minutes, preferably 30 minutes, 1 hour, 3 hours, 5 hours, 8 hours, or more, generally at the discretion of the treating physician, for the successful treatment of an acute ischemic condition. High-flow oxygen delivery apparatus (10) is usable at home, during patient transport, and, when constructed using MR-compatible materials, during MRI scans. Existing oxygen delivery systems are not made of MR-compatible materials and cannot be used during MRI scans. In addition, a portable version of high-flow oxygen delivery apparatus (10) can be manufactured; an essential feature for enabling therapy at home and for continuing therapy during patient transport.

The treatment of a patient having an ischemic condition that is not caused by or associated with injury to the head or brain, e.g., cardiovascular disease (such as myocardial infarction, heart disease, coronary artery disease, congestive heart failure, cardiac valvular disease, cardiac arrhythmia, and cardiac arrest) does not require the use of high-flow oxygen delivery apparatus (10) which has been manufactured with MR-compatible materials. For example, patients suffering from an ischemic condition caused by or associated with cardiovascular disease do not typically require an MRI scan. Thus, these patients can be administered NBO using a non-MR-compatible apparatus of the invention, if so desired.

High-flow oxygen delivery apparatus (10) can also include humidifier (16), which provides the option to humidify the oxygen delivered to the patient. Existing oxygen delivery systems do not provide the option of humidification. Humidifier (16) can provide humidified oxygen having a relative humidity of about 15 to 95% to the patient.

In addition, high-flow oxygen delivery apparatus (10) can also include one or more sensors to monitor the end-expiratory carbon dioxide levels of the patient, e.g., capnometer device (52). Capnometer sensor (60) can be placed near the mouth or capnometer sensor (64) can be placed near the nostrils to detect the levels of expired CO₂. End-tidal CO₂ levels can be measured easily from the exhaled air via a small sampling tube leading to capnometer device (52), which analyzes each breath for CO₂ content. In the absence of lung disease, the level of CO₂ measured by a capnometer correlates closely with arterial CO₂. Measurements can be obtained over long time intervals. The detection of a paCO₂ level of greater than about 40 mm Hg indicates hypoventilation.

Capnometer device (52) can further include an automatic shutoff with or without an alarm, which is activated when carbon dioxide levels present in the patient's normal expiration become too high (e.g., greater than paCO₂ of about 40 mm Hg, or an increase that is greater than 10% of the baseline value). For example, inspired oxygen therapy in patients with diminished respiratory drive (due to, e.g., chronic obstructive lung disease) can result in retained CO₂, which would be detected by the capnometer device and offers further protection to the patient during normobaric high-flow oxygen therapy.

High-flow oxygen delivery apparatus (10) can also include a device to provide oxygen or oxygen-containing gas only during the inspiratory phase, e.g., oxygen demand valve (54). Oxygen demand valve (54) provides a safe and effective means of delivering oxygen (e.g., 100% oxygen), by inhalation, to patients in need thereof at a flow rate appropriate for therapy (e.g., >10 L/min). Oxygen demand valve (54) is a simple method of providing oxygen (e.g., 100% oxygen) without the wastage of gas that occurs with free flow oxygen therapy masks.

High-flow oxygen delivery apparatus (10) can also include a device to monitor the oxygen saturation level of the patient's peripheral arterial blood, e.g., oxygen arterial saturation sensor (62)

High-flow oxygen delivery apparatus (10) is effective in improving clinical and radiological parameters of ischemia, and thus, can be used alone to treat ischemic conditions or as an adjunct therapy. Because the high-flow oxygen delivery apparatus can be modified to be portable, it can easily be used by “high risk” patients in their homes.

High-flow oxygen delivery apparatus (10) can also include an oxygen concentrator or a portable liquid oxygen (LOX) system for supplying oxygen to the patient. Oxygen concentrators operate by concentrating the oxygen already existing in the room air by eliminating the nitrogen component. Liquid oxygen systems operate by converting the liquid oxygen to gaseous oxygen within the reservoir for breathing. Liquid oxygen systems can be provided as small, lightweight portable units.

Furthermore, in the hospital setting, the oxygen source for the high-flow oxygen delivery apparatus can be provided by the hospital. Thus, in this instance, the high-flow oxygen delivery apparatus would not need to include its own oxygen cylinder, nor does it need to be portable.

### EXAMPLE 1

### Administration of Normobaric High-Flow Oxygen Therapy (NBO) to Treat Stroke

Identifying strategies to extend the thrombolysis time window is an important area of stroke research. One approach is to arrest the transition of ischemia to infarction (“buy time”) until reperfusion can be achieved. Hyperoxia might be a useful physiological therapy that slows down the process of infarction, and has shown promise in studies of myocardial infarction. Tissue hypoxia is a key factor contributing to cell death after stroke and oxygen easily diffuses across the blood-brain barrier. Moreover, oxygen has multiple beneficial biochemical, molecular and hemodynamic effects. Hyperbaric oxygen therapy (HBO) has been widely studied because it significantly raises brain tissue pO2 (ptiO2). Transient “during-therapy” clinical improvement was documented 40 years ago, and HBO proved effective in animal studies. However, the failure of 3 clinical stroke trials has reduced the enthusiasm for using HBO in stroke.

In light of the difficulties with HBO, we have begun to investigate normobaric oxygen therapy (NBO), or the delivery of high-flow oxygen via a facemask. NBO has several advantages: it is simple to administer, noninvasive, inexpensive, widely available, and can be started promptly after stroke onset (for example by paramedics). While brain ptiO2 elevation with NBO is minor as compared to HBO, the critical mitochondrial oxygen tension is extremely low and even small increases in ptiO2 might suffice to overcome thresholds for neuronal death. Recent studies indicate that brain ptiO2 increases linearly with rising concentrations of inspired oxygen, and nearly 4-fold increases over baseline have been documented in brain trauma patients treated with NBO. A recent in vivo electron paramagnetic resonance oximetry study has shown that NBO significantly increases ptiO2 in “penumbral” brain tissue. In rodents, NBO therapy during transient focal stroke attenuates diffusion-MRI (DWI) abnormalities, stroke lesion volumes, and neurobehavioral outcomes without increasing markers of oxidative stress. Based on pre-clinical results, we conducted a pilot clinical study to examine the risks and benefits of NBO in stroke.

Methods

This randomized, placebo-controlled study with blinded MRI analysis was approved by our hospital's Human Research Committee. The inclusion criteria were: (1) non-lacunar, anterior circulation ischemic stroke presenting <12 hours after witnessed symptom onset, or <15 hours after last seen neurologically intact, (2) ineligible for intravenous/intra-arterial thrombolysis, (3) National Institutes of Health Stroke Scale (NIHSS) score >4, (4) pre-admission modified Rankin scale (mRS) score <1, (5) Mean transit time (MTT)-lesion larger than DWI-lesion (perfusion-diffusion “mismatch”) with evidence for cortical hypoperfusion on MRI. To minimize time to treatment, “mismatch” was assessed during the initial MRI, using a visual estimate for >20% difference between DWI- and MTT-lesion size. The exclusion criteria were: (1) active chronic obstructive pulmonary disease, (2) >3 L/min oxygen required to maintain peripheral arterial oxygen saturation (SaO2) >95% as per current stroke management guidelines, (3) rapidly improving neurological deficits, (4) medically unstable, (5) pregnancy, (6) inability to obtain informed consent, (6) contraindication for MRI. Eligible patients were consented and randomized by opening sealed envelopes containing treatment allocation to the NBO-group (humidified oxygen via simple facemask at flow rates of 45 L/min) or the control-group (room air, or nasal oxygen 1-3 L/min if necessary to maintain SaO2>95%). NBO was stopped after 8 hours, however nasal oxygen was continued if clinically warranted.

NIHSS, mRS and Scandinavian Stroke Scale (SSS) scores were recorded after the admission MRI. NIHSS scores and MRI scans were repeated at 4 hours (range, 2.5-5.5 hours); 24 hours (range, 20-28 hours); 1 week (range, 5.5-8.5 days); and 3 months (range, 80-115 days). SSS and mRS scores were repeated at 3 months. The unblinded clinical investigator monitored patients during therapy.

Manual MRI analysis was performed by two neuroradiologists, blinded to clinical presentation, treatment group, clinical course, and medications. Stroke volumes were calculated from DWI images except for 1 week and 3 month time-points, when fluid-attenuated inversion-recovery images were used. Lesions were outlined on each axial slice using a commercially available image-analysis program (ALICE®, Perceptive Informatics, Waltham, Mass.) to yield total volumes. Reperfusion (defined as clear identification of a previously-occluded artery on MRA, or >50% decrease in MTT-lesion volume in patients without arterial “cut-off” on initial MRA) was determined on 4 hour and 24 hour MRIs. Post-ischemic hemorrhage was ascertained on 24 hour gradient-echo MRIs.

Automated MRI analysis was performed to determine the fate of individual voxels on apparent diffusion coefficient (ADC) maps, as per their change in signal intensity above or below a threshold of 600×10⁻⁶ mm²/s (approximately 45% of normal) from baseline to the 4 hour and 24 hour time-point. Voxels with signal intensity constantly above-threshold were considered “never-abnormal”; remaining voxels were grouped as follows: (1) No reversal, signal intensity below-threshold at all time-points; (2) Temporary early reversal, signal intensity below- threshold at baseline, improving to an above-threshold value at 4 hours, but reverting at 24 hours; (3) Sustained early reversal, signal intensity below-threshold at baseline, improving to an above-threshold value at 4 hours and 24 hours; (4) Late reversal, signal intensity below-threshold at baseline and 4 hours, improving to an above-threshold value at 24 hours; and (5) Progression to ischemia, signal intensity above-threshold at baseline, worsening to a below-threshold value at 4 hours or 24 hours. We further analyzed voxels with “sustained early reversal” for “late secondary decline” on the 1 week MRI.

For each patient, outlines of the baseline MTT-lesion were transferred onto co-registered perfusion maps at each time-point, and relative cerebral blood volume, blood flow, and MTT (rCBV, rCBF, rMTT) values were calculated within these regions after normalizing to a region of gray matter in the contralateral hemisphere.

The pre-specified primary outcome was a comparison of DWI-lesion growth at 4 hours between groups. Secondary outcomes were changes in NIHSS scores and perfusion parameters at 4 hours, the percentage of ADC voxels undergoing reversal at 4 hours or 24 hours, brain hemorrhage at 24 hours, and 3 month stroke lesion volumes, NIHSS and mRS scores. We initially planned to enroll 40 patients in this pilot study in order to allow formal power calculations. The interim analysis showed positive results, which are presented herein.

Imaging Technique

The following MRI sequences were performed: sagittal T1, axial DWI with apparent diffusion coefficient (ADC) maps, T2, fluid-attenuated inversion recovery (FLAIR), and gradient-echo (GRE). In addition, perfusion-MRI (PWI) and head MR-angiography (MRA) were performed with the admission, 4 hour and 24 hour MRI scans.

Imaging was performed using 1.5 T (General Electric, Waukesha, Wis.) clinical MRI systems. The FLAIR, and GRE, series had the following relevant parameters: 24 cm field of view, 7 mm-thick axial-oblique slices aligned with the anterior-posterior commissure, 20 slices contiguous, interleaved, and co-localized. Diffusion-weighted images were acquired using a FoV=220 mm, 23 slices, thickness=6 mm, gap=1 mm, TR=7.5 s, TE=99 ms, acquisition matrix 128×128, and with both b=0 s/mm²and b=1,000 S/mm² in 6 diffusion gradient directions, number of averages=3. Isotropic diffusion weighted (DWI) images and apparent diffusion coefficient (ADC) images were automatically calculated. FLAIR and T2-weighted imaging was done with a fast-spin echo (FSE) sequence having TR/TE=9,000/85 msec, TI=1,750 msec and a 256×128 matrix; and using GRE T2* imaging with TR/TE=800/20 msec and a 256×192 matrix. PWI images were obtained using the standard bolus passage of contrast method by injecting gadolinium (0.1 mmol/kg dose via power injector) with gradient-echo echo planar imaging, 11 slices, FoV=220 mm, TR=1.5 s, TE of 54 ms; 46 measurements, matrix of 128×128. Maps of cerebral blood volume (CBV), cerebral blood flow (CBF), and mean transit time (MTT) were calculated as described previously. We did not create “threshold” MTT maps based on any time-delay. MTT lesion volumes were computed by manually outlining regions of hyperintensity on MTT maps, using a commercially available image analysis program (ALICE®). The three-dimensional time-of-flight magnetic resonance angiography (MRA) consisted of a single slab, approximately 7 cm thick, positioned over the circle of Willis, coplanar to the other slice prescriptions. The relevant imaging parameters were TR/TE=39/6.9 msec, 25 degree flip-angle, FOV=24×18 cm with a matrix=224×160 for an in-plane resolution of approximately 1 mm, reconstructed to 92 axial images, 1.6 mm thick with a 0.8 mm overlap, for a total acquisition time of 3 minutes and 11 seconds. The MRA source images were post-processed into maximum intensity projection images using standard software tools.

Automated MRI analysis was performed using Matlab (The MathWorks, Inc., Natick, Mass.). All images were subjected to a motion-correction algorithm and diffusion-tensor images were corrected for eddy-current distortions using FLIRT. Images were co-registered to the baseline MRI study to allow voxel-by-voxel analysis of tissue fate over time.

Statistical Analysis

SPSS® for Windows (v11.0; SPSS, Chicago, Ill.) was used for the “intention to treat” statistical analysis. All values are reported as median (range), or mean±SD. For inter-group comparisons we applied the Student t-test, Mann-Whitney U test, or Fisher's exact test; for intra-group comparisons we applied the paired t-test or Wilcoxon rank-sum test as appropriate. P<0.05 was considered significant.

Results

We randomized 9 patients to the NBO-group and 7 to the Control-group. No patient developed hypoventilation. None complained of discomfort from the facemask. Mean blood glucose, mean arterial BP at baseline, 4 hours and 24 hours, and anticoagulant and antiplatelet use, were not significantly different between groups. ABG was drawn for clinical reasons in three patients: the paO2 (mm Hg) was 368 and 420 in two NBO patients, and 99 in one Control patient. Table 1 shows patient characteristics.

TABLE 1
Patient Data

| | Hyperoxia | Controls |
|---|---|---|
| Characteristic | (n = 9) | (n = 7) |
| Age, years (mean, range) | 67 (37-88) | 70 (49-97) |
| Female | 5 (56%) | 4 (57%) |
| Stroke Etiology | | |
| Cardioembolic | 6 | 5 |
| ICA atherosclerosis/thrombosis | 3 | 0 |
| ICA dissection | 0 | 1 |
| Cryptogenic embolism | 0 | 1 |
| Intravenous heparin on day 1 | 5 (56%) | 5 (71%) |
| Stroke Scale Scores (median, range) | | |
| Admission NIHSS | 14 (4-22) | 11 (8-21) |
| 4-hour NIHSS | 12 (2-15) | 13 (10-26) |
| 24-hour NIHSS | 6 (4-16) | 15 (11-26) |
| 1-week NIHSS | 6 (0-22) | 14 (7-23) |
| 3-month NIHSS | 3 (0-19) | 13 (1-19) |
| Admission Scandinavian Stroke Scale | 27 (6-55) | 32 (2-39) |
| 3-month Scandinavian Stroke Scale | 47 (16-60) | 32 (30-56) |
| 3-month mRS (mean ± SD) | 3.2 ± 2.2 | 4.1 ± 1.6 |
| MRI Characteristics (median, range) | | |
| Time Intervals | | |
| Onset to MRI-1 (hours) | 7.4 (1.6-13.4) | 6.8 (3.5-8.9) |
| MRI-1 to MRI-2 (hours) | 4 (2.6-4.7) | 4.5 (3.5-5.7) |
| MRI-1 to MRI-3 (hours)* | 24.4 (21.3-26.5) | 25 (22.5-27.7) |
| MRI-1 to MRI-4 (days)* | 6.6 (3.7-8.2) | 6.2 (4.0-9.9) |
| MRI-1 to MRI-5 (days)* | 99 (54-106) | 116 (107-152) |
| Post-ischemic hemorrhage on MRI-2 | 1 (asymptomatic) | 1 (fatal) |
| Post-ischemic hemorrhage | 4 (50%) | 1 (17%) |
| on MRI-3* | | |
| Reperfusion | | |
| MRI-1 to MRI-2 | 0 (0%) | 1 (14%) |
| MRI-2 to MRI-3* | 4 (50%) | 0 (0%) |

*Excluding one patient per group with post-ischemic hemorrhage.

Soon after the admission MRI, one control patient developed a massive brain hemorrhage and died; one NBO patient developed an asymptomatic brain hemorrhage temporally associated with a supra-therapeutic partial thromboplastin time from IV heparin treatment. Individual patient data is shown in Table 2.

TABLE 2
(A) Individual Patient Data: Serial NIHSS Scores, and relative DWI/FLAIR/MTT lesion volumes

| Patient | Baseline | | | 4 hours | | | 24 hours | | | 1 week | | 3 months | |
|---|---|---|---|---|---|---|---|---|---|---|---|---|---|
| No. | NIHSS | DWI | MTT | NIHSS | DWI | MTT | NIHSS | DWI | MTT | NIHSS | FLAIR | NIHSS | FLAIR |
| NBO-1 | 14 | 100 | 100 | 12 | — | — | 7 | — | — | — | — | — | — |
| NBO-2 | 4 | 100 | 100 | 2 | 76 | 195 | 5 | 116 | 138 | 10 | 337 | 7 | 119 |
| NBO-3 | 8 | 100 | 100 | 4 | 66 | 83 | 4 | 72 | 23 | 0 | 105 | 0 | 57 |
| NBO-4 | 18 | 100 | 100 | 15 | 96 | 123 | 4 | 122 | 49 | 4 | 147 | — | — |
| NBO-5 | 22 | 100 | 100 | — | 75 | 86 | — | 112 | 80 | 22 | 232 | 19 | 286 |
| NBO-6 | 12 | 100 | 100 | 4 | 89 | 56 | 4 | 120 | 113 | 5 | 158 | 3 | 136 |
| NBO-7 | 19 | 100 | 100 | 15 | 64 | 82 | 16 | 145 | 61 | 12 | 186 | 8 | 169 |
| NBO-8 | 16 | 100 | 100 | 12 | 110 | 132 | 11 | 175 | 30 | 7 | 228 | 2 | 224 |
| NBO-9 | 12 | 100 | 100 | 11 | 127 | 84 | 8 | 146 | 35 | 2 | 194 | 2 | 121 |
| Ctrl-1 | 10 | 100 | 100 | — | — | — | — | — | — | — | — | — | — |
| Ctrl-2 | 11 | 100 | 100 | 15 | 123 | 159 | 18 | 172 | 53 | 17 | 215 | 12 | 173 |
| Ctrl-3 | 11 | 100 | 100 | 12 | 149 | 97 | 11 | 175 | 99 | 7 | 214 | 1 | 130 |
| Ctrl-4 | 8 | 100 | 100 | 10 | 144 | — | 14 | 87 | 156 | 13 | 108 | 13 | 129 |
| Ctrl-5 | 12 | 100 | 100 | 12 | 165 | 135 | 16 | 482 | 57 | 14 | 429 | — | — |
| Ctrl-6 | 21 | 100 | 100 | 26 | 218 | 36 | 26 | 1327 | 37 | 23 | 2202 | 19 | — |
| Ctrl-7 | 14 | 100 | 100 | 13 | 95 | — | 13 | 106 | 90 | 14 | 128 | — | — |

Median NHSS, SSS and mRS scores are presented in Table 1, and inter-group comparisons of mean NIHSS scores in FIG. 4A. In the NBO-group, clinical improvement was noted as early as 15-20 minutes after starting the 8 hour hyperoxia therapy. As compared to baseline, mean NIHSS scores were significantly lower at 4 hours (p=0.016), 24 hours (p=0.03) and 3 months (p=0.03).

All patients had ICA and/or proximal MCA occlusion with substantial perfusion-deficits (MTT-lesion volume >90 cc in 13 of 16 patients). Mean MTT (NBO, 125.9±65 cc versus control, 130.5±81 cc, p=0.9) and DWI (NBO, 29.3±22 cc; control, 27.1±39 cc, p=0.89) lesion volumes were comparable at baseline. At 4 hours, reperfusion was evident in one control patient; however, mean MTT-lesion volumes were not significantly different between groups (p=0.4). At 24 hours, 4 NBO-treated patients but no additional control patients showed reperfusion on MRI, and mean MTT-lesion volumes were significantly lower than baseline in the NBO-group (87.8±48 cc versus 125.9±65 cc, p=0.04).

Asymptomatic petechial hemorrhages were evident on 24 hour MRI scans in 4 NBO patients and in 1 control patient (p=0.6); were located in the deep MCA territory; and were associated with reperfusion (3 patients) and prior microbleeds (1 patient).

At 4 hours (during therapy), relative DWI-lesion volumes decreased in 6 NBO-treated patients, with >20% reduction in 3 patients. DWI reversal was most evident in the lesion periphery (FIG. 5) and was not associated with regions of tissue reperfusion. Among controls, only 1 patient had a smaller DWI volume at 4 hours, and the reduction was minor (5%). Mean relative DWI-volumes were significantly smaller in the NBO-group as compared to controls at 4 hours (87.8±22% versus 149.1±41%, p=0.004), but not significantly different at 24 hours, 1 week and 3 months (FIG. 4B). Penumbral salvage was significantly higher in the NBO-group at 4 hours (FIG. 4C).

Voxels showing temporary and sustained ADC reversal were located mainly in gray- and white-matter regions in the lesion periphery (FIG. 6A). The NBO-group tended to have a higher average percentage of voxels undergoing “temporary early reversal” (FIG. 6B). While the percentage of “sustained early reversal” voxels was 3-fold higher in the NBO-group than controls, the difference was not statistically significant. Temporary or sustained ADC reversal in voxels totaling a volume >1.5 cc was observed in 6 NBO and one control patient (p=0.1). There was no significant difference in the percentage of voxels with “late secondary decline.”

Mean rCBV and mean rCBF increased significantly from baseline to 4 hours and 24 hours in the NBO group, but not in the control group; mean rMTT showed no significant change over time in either group (FIG. 7).

Discussion

In this study, NBO started within 12 hours after onset of ischemic stroke transiently improved clinical function and MRI parameters of ischemia. Treatment benefit was most evident at 4 hours (during therapy) when there was no evidence for arterial recanalization—a factor associated with DWI improvement. However, some benefit persisted at 24 hours and at 1 week, perhaps related to subsequent reperfusion and/or direct effects of oxygen therapy. This imaging pattern is believed to indicate the presence of penumbral tissue, or the target tissue for neuroprotection. An increasing number of stroke therapeutic trials using this selection criterion are reporting success. While further studies are mandated to investigate NBO's therapeutic time window, optimum duration, and effects in different stroke subtypes, the results of the present study indicate that (1) by delaying ischemic necrosis, NBO has utility as a stroke therapy (and other forms of acute ischemic conditions), particularly as an adjunctive therapy that widens the time window for reperfusion and other neuroprotective therapies, and (2) multiparametric MRI can effectively quantify neuroprotection.

The concordance between changes in clinical and MRI measurements, and their temporal correlation with NBO exposure (FIG. 4A), provides substantial evidence that NBO is beneficial if administered after acute hemispheric stroke.

Hyperoxia induces vasoconstriction in normal brain tissue. However in this study, hyperoxia increased rCBV and rCBF within areas of initial MTT abnormality, consistent with results of our rodent experiments. Prior clinical studies have documented paradoxical vasodilatation in the ischemic brain after oxygen exposure. Overall, these data suggest a novel neuroprotective mechanism for hyperoxia: shunting of blood from non-ischemic to ischemic brain tissues.

Hyperoxia therapy can depress respiratory drive in patients with chronic lung disease, decrease cardiac output, and increase systemic vascular resistance. Decades of research have emphasized the harmful tissue effects of oxygen free radical injury. Our pre-clinical studies indicate that hyperoxia's benefit in reducing infarct volume, outweighs the risk of enhanced free radical injury. Similarly, in this study, we found no evidence for clinical or radiological worsening with NBO. Four NBO-treated patients developed asymptomatic petechial post-ischemic hemorrhage, raising the possibility that oxygen worsened reperfusion injury. However, such hemorrhages have been correlated with successful recanalization (as in 3 of 4 patients in this study), reduced infarct size, and better clinical outcomes.

### EXAMPLE 2

### Administration of Normobaric High-Flow Oxygen Therapy (NBO) to Treat Cardiac Ischemia

Normobaric high-flow oxygen therapy (NBO) can be administered to a patient who presents with severe chest pain. An electrocardiogram (EKG) can be performed on the patient to confirm characteristics of acute cardiac ischemia. NBO is started immediately upon onset of symptoms or diagnosis. Oxygen is delivered at a flow rate of 25 L/min or greater, and preferably 40 L/min, and at a concentration of between 95% and 100% oxygen. The patient is administered NBO on route to the hospital and throughout the hospital stay (e.g., the patient can be administered thrombolytics and/or angioplasty and stenting for his severely occluded or stenotic coronary arteries concurrently with NBO). The administration of NBO may persist upon discharge of the patient from the hospital.

Normobaric high-flow oxygen therapy (NBO) can be administered to the patient for 8 hours or more, generally at the discretion of the treating physician. NBO can also be administered until reperfusion of the organ occurs, although therapy can continue beyond this point.

The patient's outcome (size of myocardial infarct and subsequent cardiac arrhythmia and development of congestive heart failure) will be improved when compared to a similar patient who did not receive NBO. The improvement in the patient administered NBO occurs because oxygen, administered at normobaric pressure, has effectively penetrated ischemic cardiac tissue (both via the arterial supply to the heart, and superoxygenated blood in the ventricles).

### EXAMPLE 3

### Administration of Normobaric High-Flow Oxygen Therapy (NBO) to Treat Brain Trauma

Normobaric high-flow oxygen therapy (inspired O₂ (FIO₂) concentration 100%) can be administered in the treatment of patients with traumatic brain injury (TBI) caused by, e.g., an automobile accident. For example, a patient with TBI can be treated for 2 to 24 hours or more with 50% to 100% FIO₂, preferably about 100% FIO₂, starting immediately after, or at least within 6 hours of, admission to the hospital. The treated patient can be evaluated using Glasgow Coma Scale scores after resuscitation and for intracranial pressure within the first 8 hours after admission. The patient can be monitored with the aid of intracerebral microdialysis and tissue O₂ probes. NBO can result in significant improvement in intracranial pressure, in the level of biochemical markers in the brain, and the level of various markers in the blood (e.g., glucose levels, glutamate and lactate levels, and lactate/glucose and lactate/pyruvate ratios), as compared with the baseline measures. Patients receiving NBO would have better long-term clinical outcomes than untreated patients.

What is claimed is:

1. An oxygen delivery apparatus comprising:
a) a source of oxygen; and
b) means for delivering, to the respiratory system of a human patient, oxygen from said source of oxygen at a flow rate of 10 L/min or greater at normal or near-normal atmospheric pressure, wherein said apparatus is adapted such that the apparatus is:
i) portable, and
ii) MRI-compatible.

2. The apparatus of claim 1, wherein said source of oxygen comprises 90% to 100% oxygen.

3. The apparatus of claim 2, wherein said source of oxygen comprises 100% oxygen.

4. The apparatus of claim 1, wherein said flow rate is between 25 L/min and 60 L/min.

5. The apparatus of claim 4, wherein said flow rate is 40 L/min.

6. The apparatus of claim 1, wherein said apparatus is manufactured using non-metallic, non-ferromagnetic, or non-paramagnetic material.

7. The apparatus of claim 6, wherein said material is plastic or silicon.

8. The apparatus of claim 1, wherein said means for delivering oxygen to said patient comprises a facemask coupled to said source of oxygen.

9. The apparatus of claim 1, further comprising humidifying means for humidifying the oxygen from said source.

10. The apparatus of claim 1, further comprising end-tidal capnometer and capnometer sensor means.

11. The apparatus of claim 10, wherein said end-tidal capnometer further comprises an alarm and automatic shutoff that prevents the flow of oxygen to said patient, both of which activate when said capnometer sensor means detects expiratory partial pressure of arterial carbon dioxide (paCO₂) levels of greater than 40 mm Hg or greater than 10% or more of baseline values.

12. The apparatus of claim 1, further comprising gas demand valve means that allows the flow of oxygen during inspiration of the patient.

13. A method of reducing acute ischemic damage in a human patient comprising delivering oxygen to the respiratory system of said patient at a flow rate of 25 L/min or greater at normal or near-normal atmospheric pressure.

14. The method of claim 13, wherein said acute tissue damage is caused by subarachnoid hemorrhage, brain hemorrhage, brain trauma, head injury, a seizure, a headache disorder, cardiovascular disease, tissue organ engraftment rejection, sequelae of ischemic reperfusion injury, retinal ischemia, gastrointestinal ischemia, or peripheral ischemia.

15. The method of claim 14, wherein said cardiovascular disease is myocardial infarction, heart disease, coronary artery disease, congestive heart failure, cardiac valvular disease, cardiac arrhythmia, or cardiac arrest.

16. The method of claim 14, wherein said retinal ischemia is caused by diabetic retinopathy, central retinal artery or vein occlusion, stenosis of the carotid artery, retinal detachment, retinal tearing, or sickle cell retinopathy.

17. The method of claim 14, wherein said gastrointestinal ischemia is ischemic bowel, ischemic colitis, or mesenteric ischemia.

18. The method of claim 14, wherein said headache disorder is migraine or cluster headache.

19. The method of claim 14, wherein said peripheral ischemia is acute peripheral vascular ischemia, atherosclerosis, peripheral arterial occlusive disease, thromboembolic disease, or thromboangiitis obliterans (Buerger's disease).

20. The method of claim 13, wherein said acute ischemic damage occurs in the brain, heart, peripheral nervous system, bowels, kidney, or retina.

21. The method of claim 13, wherein said method delivers 95% oxygen to the respiratory system.

22. The method of claim 21, wherein said method delivers 100% oxygen to the respiratory

23. The method of claim 13, wherein said oxygen is delivered to said patient using delivery means.

24. The method of claim 23, wherein said delivery means comprises a facemask coupled to a source of oxygen.

25. The method of claim 13, wherein said flow rate is between 25 L/min and 60 L/min.

26. The method of claim 24, wherein said flow rate is 40 L/min.
